(19) United States
(12) Patent Application Publication
(10) Pub. No.: US 2004/0053221 A1
(43) Pub. Date: Mar. 18, 2004

(54) Methods for analyzing antibody response in an individual to major surface glycoprotein (Msg), immunological assays, and recombinant MsgA, MsgB and MsgC fragments

(76) Inventors: Peter D. Walzer, Loveland, OH (US); Kieran R. Daly, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 10/464,261

(22) Filed: Jun. 18, 2003

Related U.S. Application Data
(60) Provisional application No. 60/389,632, filed on Jun. 18, 2002.

(51) Int. Cl. C12Q 1/70; G01N 33/554; G01N 33/569

(52) U.S. Cl. 435/5; 435/7.32

(57) ABSTRACT

Methods of analyzing antibody response in an individual to major surface glycoprotein (Msg) comprise analyzing a pattern of reactivity of a sample with a recombinant Msg fragment. Immunological assays for analyzing antibody response to major surface glycoprotein (Msg) in an individual comprise recombinant Msg fragments. Recombinant MsgA, MsgB, and MsgC fragments are also provided.

### RELATED APPLICATION

This application claims priority under 35 U.S.C. §119 of U.S. Provisional Application Serial No. 60/389,632 filed Jun. 18, 2002.

### GOVERNMENT INTERESTS

This invention was made, at least in part, with funds from the Federal Government, awarded through grant numbers: National Institute of Health AACTG.27.Pc01, National Institute of Health Contract AI-753A, Veterans Administration Research Grant, and National Institute of Health Research Grant R01 HL-64570, SP-A.

### FIELD OF THE INVENTION

The present invention is directed toward methods of analyzing antibody response to major surface glycoprotein (Msg) in an individual. The present invention is also directed to immunological assays for analyzing antibody response to major surface glycoprotein (Msg) in an individual. The present invention is further directed to recombinant MsgA, MsgB and MsgC fragments.

### BACKGROUND OF THE INVENTION

Pneumocystis jiroveci (P. jiroveci), a fungal opportunistic pathogen of humans previously known as Pneumocystis carinii fsp. hominis, is the causative agent of Pneumocystis pneumonia (PcP), a leading cause of serious illness in immunocompromised patients. The use of potent anti-human immunodeficiency virus (HIV) drugs has dramatically reduced the frequency of opportunistic infections, including P. jiroveci, in HIV⁺ patients. Furthermore, the use of highly active anti-retroviral therapy (HAART) is associated with reconstitution of the immune system. Although the effects of HAART are generally measured by the increase in T cell numbers, data on reconstitution of organism-specific immune responses have been limited.

There is a high prevalence of anti-P. jiroveci antibodies in normal healthy adults, with greater than 70% of sera positive for reactivity to P. jiroveci antigens. This level of reactivity is consistent with widespread exposure to P. jiroveci organisms, which happens during childhood. Serum antibodies reactive with P. jiroveci antigens are commonly found in children of less than 4 years of age. It is known that many proteins of P. jiroveci can be recognized by serum antibodies, but little information exists on the responses to specific P. jiroveci antigens, how those responses change over time and whether these responses are protective. This is, in part, due to the fact that the antigen preparations used in previous studies were crude homogenates from human or rodent lungs, and did not allow the identification of specific antigenic proteins. Furthermore, sera isolated in different parts of the world exhibited different patterns of reactivity to P. jiroveci antigens in Western blot analysis, suggesting that there may be variability between different P. jiroveci preparations, or that there are geographic differences in reactivity to P. jiroveci antigens.

One of the P. jiroveci proteins that is consistently recognized by serum antibodies and helper T cells is a 95 kD protein called the major surface glycoprotein (Msg) or glycoprotein A (gpA). This antigenic protein shares epitopes with the 120 kD antigen of rodent Pneumocystis carinii (P.c.), elicits protective B and T cell responses, and plays an important role in the interaction of Pneumocystis organisms with the host lung. Analysis of antibody reactivity to Msg has been hampered by the crude antigen preparations described above, and the inherently diverse nature of Msg molecules. For example, native Msg is recognized in only 30-40% of serum specimens from healthy individuals.

Accordingly, there is a need to simplify antigen preparations to analyze the immune response to a single Msg fragment. The identification of antigenic epitopes on a single isoform of Msg would allow tracking of the response to that specific epitope during a Pneumocystis infection, in convalescence following an episode of PcP, and in evaluating the antigen specific immune reconstitution of HIV+patients following HAART.

### SUMMARY OF THE INVENTION

Accordingly, it is an object to provide methods to simplify antigen preparations to analyze the immune response to a single Msg fragment. In accordance with one aspect of the invention, there are provided methods of analyzing antibody response in an individual to major surface glycoprotein (Msg). The methods comprise analyzing a pattern of reactivity of a sample with a recombinant Msg fragment.

In accordance with another aspect of the invention, there are provided immunological assays for analyzing antibody response to major surface glycoprotein (Msg) in an individual. The immunological assays comprise recombinant Msg fragments.

In accordance with yet another aspect of the invention a recombinant MsgA fragment is provided. In accordance with a further aspect of the invention a recombinant MsgB fragment is provided. In accordance with yet a further aspect of the invention a recombinant MsgC fragment is provided.

### DETAILED DESCRIPTION OF THE FIGURES

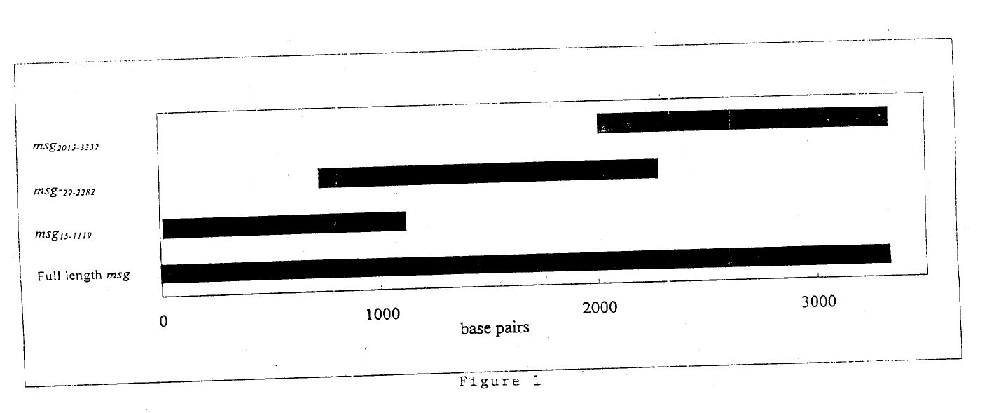

FIG. 1 illustrates Msg recombinants as compared with the full length Msg gene.

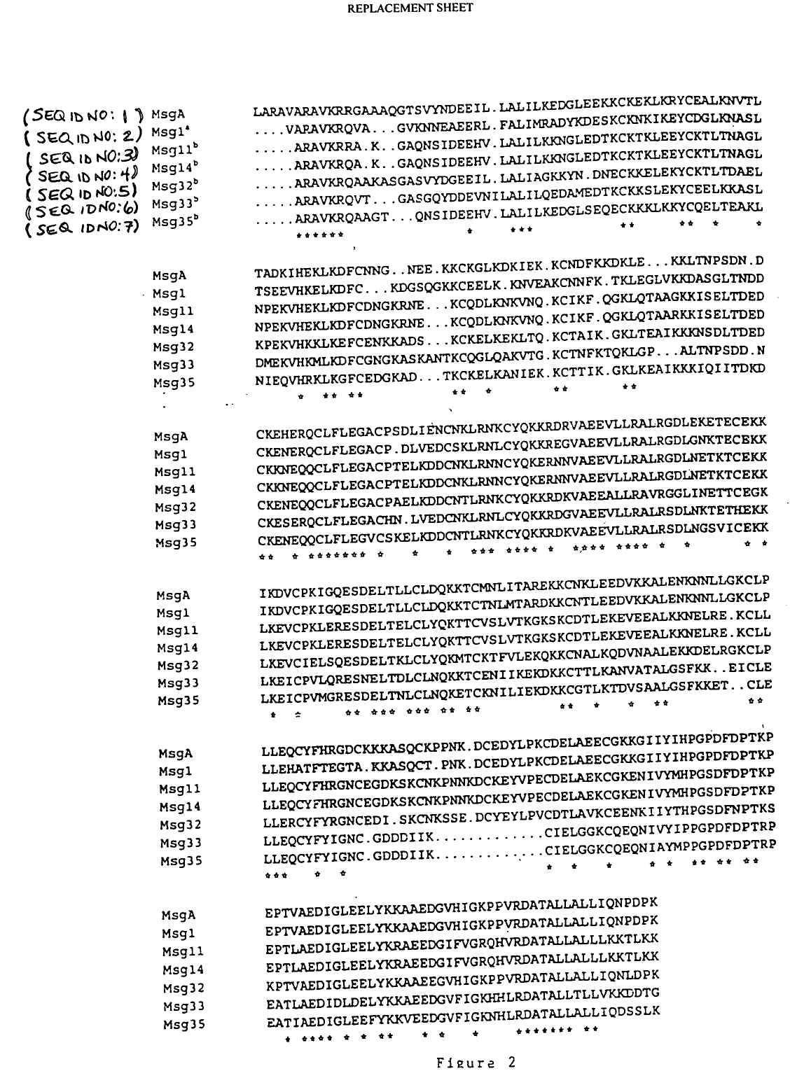

FIG. 2 shows a comparison of the deduced amino acid sequences of MsgA and the corresponding sequences of the known Msg gene products, wherein “*” indicates identity and “.” indicates space is introduced for optimal alignment.

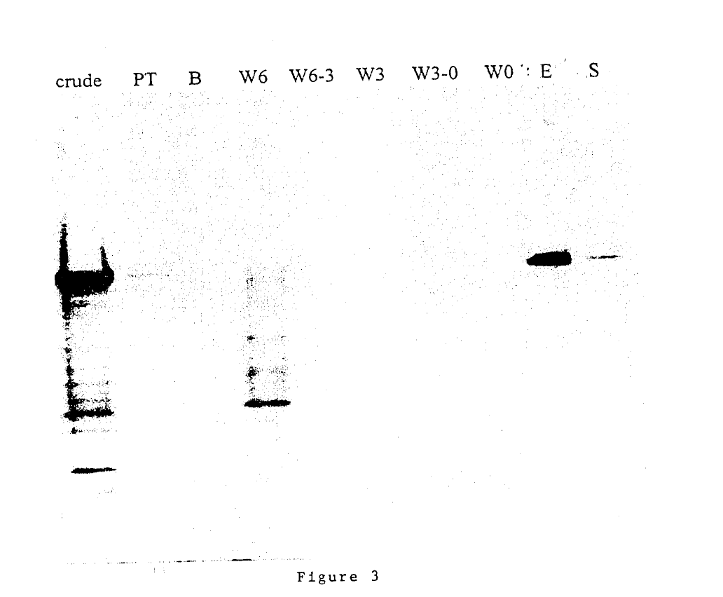

FIG. 3 shows a purification profile of MsgB. Inclusion bodies containing MsgB were dissolved in urea (crude) and purified on HIS tag affinity columns, wherein PT is pour though, B is binding buffer, W is wash solution with the numerical values representing the molar strength of urea in the solution, E is protein eluted from the column and S is the protein stripped from the column at the end of the purification process.

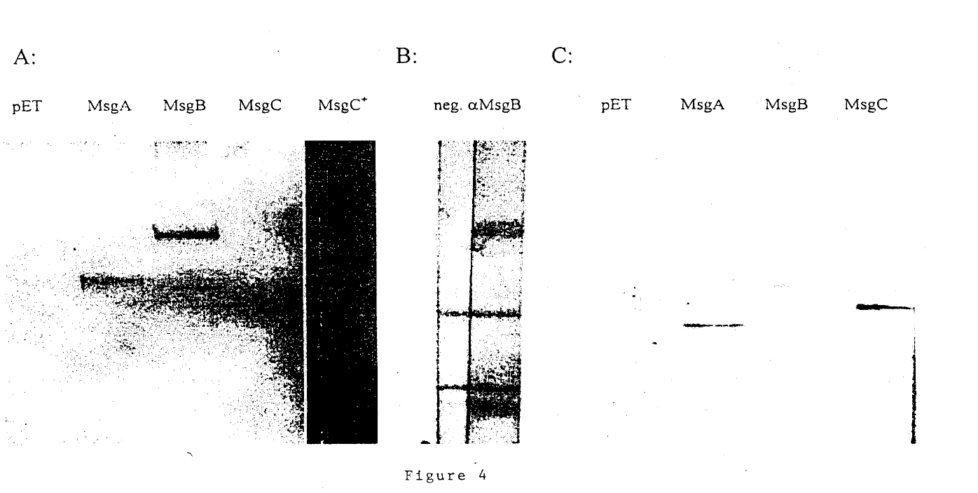

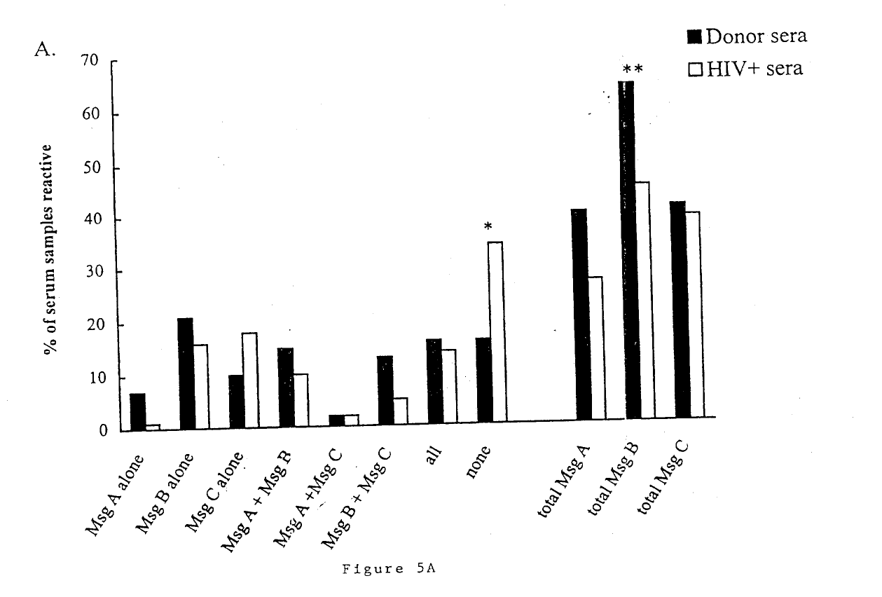

FIG. 4 shows specificity of the Msg constructs as shown by antibody reactivity with MsgA, MsgB and MsgC but not with the control (pET). A: Reactivity of a polyclonal rabbit antiserum, raised against a cell wall preparation of Pneumocystis jiroveci, for the Msg constructs. Clear reactivity to MsgC was seen after increase development time (MsgC⁺) B: Rabbit antibodies eluted from MsgB (aMsgB) but not antibodies eluted from an irrelevant protein (neg.) react with a 95 kD protein (*) in a crude homogenate of infected human lung. C: the reactivity of human serum for each of the Msg recombinants.

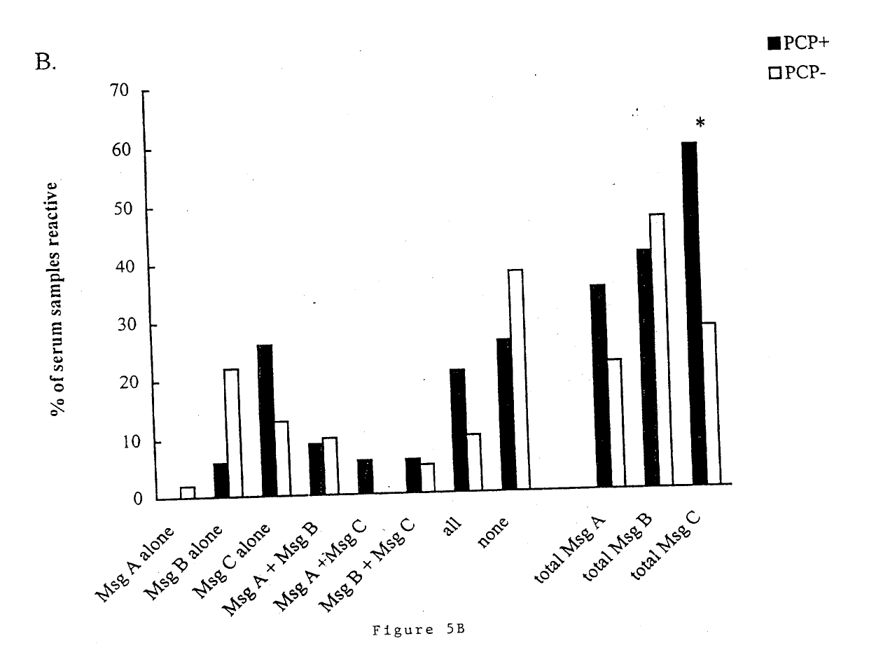

FIG. 5A shows the frequency of reactivity of blood donor sera and sera from human immunodeficiency virus (HIV)⁺ patients to the Msg fragments. P=0.003* and 0.0086** when HIV⁻ and HIV⁺sera are compared.

FIG. 5B shows the frequency of reactivity of HIV+donors with prior PcP and those without exposure to PcP to the Msg fragments. P=0.0046* when PcP⁻ and PcP⁺ sera are compared.

### DETAILED DESCRIPTION OF THE INVENTION

Pneumocystis jiroveci (P. jiroveci), a fungal opportunistic pathogen of humans previously known as Pneumocystis carinii fsp. hominis, is the causative agent of Pneumocystis pneumonia (PcP), a leading cause of serious illness in immunocompromised patients. As noted above, one of the P. jiroveci proteins that is consistently recognized by serum antibodies and helper T cells is a 95 kD protein called the major surface glycoprotein (Msg) or glycoprotein A (gpA). This antigenic protein shares epitopes with the 120 kD antigen of rodent Pneumocystis carinii (P.c.), elicits protective B and T cell responses, and plays an important role in the interaction of Pneumocystis organisms with the host lung.

Msg is a family of related proteins that is encoded by multiple genes in the Pneumocystis genome. While not wishing to be bound by theory, it appears that only one Msg is expressed at a given time, suggesting that Pneumocystis species may evade the host immune system by switching the isoform of the protein expressed. Such antigenic variation is commonly used by microorganisms such as trypanosomes and the spirochete Borrelia. Analysis of antibody reactivity to Msg has been hampered by the crude antigen preparations described above and the inherently diverse nature of Msg molecules. For example, native Msg is recognized in only 3040% of serum specimens from healthy individuals.

The present inventors have found that identification of antigenic epitopes on a single isoform of Msg allow tracking of the response to that specific epitope during a Pneumocystis infection, in convalescence following an episode of PcP, and in evaluating the antigen specific immune reconstitution of HIV⁺ individuals following HAART. Accordingly, the inventors have generated a panel of recombinant Msg fragments that correspond to a single Msg molecule for use as a standardized set of reagents in immunological assays. The inventors have characterized the specificity of the fragments, and have further analyzed the patterns of reactivity of healthy and HIV⁺ serum samples to the recombinants.

In accordance with these findings, the present invention is directed to methods for analyzing antibody response in an individual to major surface glycoprotein (Msg). The methods comprise analyzing a pattern of reactivity of a sample with a recombinant Msg fragment. Additionally, the present invention is also directed to immunological assays for analyzing antibody response to major surface glycoprotein (Msg) in an individual. The immunological assays comprise recombinant Msg fragments. Furthermore, the present invention is directed to recombinant MsgA, MsgB and MsgC fragments.

In this work, the inventors have generated three overlapping recombinant fragments of human P. jiroveci-derived Msg for use as standardized reagents in immunological assays. Each recombinant represents a single Msg molecule which can be analyzed independently. As used herein, “fragment” is intended to refer to a sequence of nucleic acids and/or amino acids encoding DNA, RNA, protein or combinations thereof. One skilled in the art will appreciate the various techniques for generating a recombinant Msg fragment, any of which may be employed herein. In one embodiment, recombinant Msg fragments are generated by cloning More specifically, oligonucleotides are designed based on the known sequence of P. jiroveci Msg, and are used in PCR to generate three overlapping fragments of a Msg gene. The sequences of the oligonucleotides used are as follows: 5′ AACTACCTTTAAAACTTTCAA 3′ (forward), 5′ TTAGGATCTGGATTCTGA 3′ (reverse) to generate msg₁₅₋₁₁₁₉, 5′ CGAAGCTTATGACTGCGCGAG 3′ (forward), 5′ TCCTCGGAGCTCTACTTTGAG 3′ (reverse) to generate msg₇₂₉₋₂₂₈₂, 5′ TTCCAAAACGCTACGTGTAA 3′ (forward), 5′ TCAATTGATGCTGAAGAGATG 3′ (reverse) to generate msg₂₀₁₅₋₃₃₃₂. The sequence of the PCR products is confirmed, and they are cloned into the pET30 expression vector (Novagen, Madison, Wis.) in the correct reading frame and expressed in E. coli. The 3 recombinant fragments are called MsgA, MsgB and MsgC which are encoded by the gene fragments msg₁₅₋₁₁₁₉, msg₇₂₉₋₂₂₈₂ and msg₂₀₁₅₋₃₃₃₂, respectively.

Given the difficulty in obtaining sufficient quantities of antigens from P. jiroveci, and the inherent variability in Msg isotypes, these generated Msg fragments represent a unique opportunity to analyze immune responses to a single Msg molecule. The usefulness of these fragments for serological studies is underscored by the fact that each fragment can be recognized in a specific manner by both human and rabbit serum antibodies.

In order to analyze antibody response of an individual with major surface glycoprotein (Msg), a sample is obtained from an individual. As used herein, “individual” is intended to refer to an animal, including but not limited to humans, mammals, and rodents. Samples include, but are not limited to blood samples, tissue samples, serum samples, or combinations thereof. The obtained sample of the individual is contacted with a recombinant Msg fragment and a pattern of reactivity is analyzed. As used herein, “pattern of reactivity” is meant to refer to the representation of molecules, including but not limited to genes, proteins or combinations' thereof, in an antibody response. One skilled in the art will appreciate the various techniques for analyzing a pattern of reactivity, any of which may be employed herein.

In a further embodiment, the step of analyzing the pattern of reactivity comprises the steps of determining the pattern of reactivity exhibited by the sample with the recombinant Msg fragment and comparing the pattern of reactivity to a predetermined pattern of reactivity. One skilled in the art will appreciate the various techniques for determining the pattern of reactivity, any of which may be employed herein. In one embodiment, the pattern of reactivity is determined by Western Blot analysis. As shown in the Example, the inventors have analyzed the patterns of healthy and HIV⁺ serum samples to the recombinant Msg fragments.

As further illustrated in the Example, western blot analysis of 95 randomly obtained serum samples from anonymous healthy donors demonstrates that each of the constructs can be recognized by human serum. Eighty of the 95 samples (84%) reacted with at least one of the recombinant Msg fragments. MsgB is recognized by 65% of the sera tested, whereas MsgA and MsgC are recognized by 40% and 41% of the sera respectively. Seventy-six percent (29/38) of the sera that recognizes MsgA and 69% (27/39) of the sera that recognizes MsgC, also recognizes MsgB. While not wishing to be bound by theory, this data suggests that MsgB may contain more commonly recognized epitopes.

This high level of reactivity in a healthy population of adults is consistent with prior exposure to Msg as an antigen, probably as a consequence of early childhood exposure to P. jiroveci. Further, while the inventors do not wish to be bound by theory, it is interesting that such a large percentage of healthy samples reacted positively with the recombinant fragments (84%), because it suggests that either the variability in Msg epitopes recognized by B cells is restricted and biased towards cross-reactive epitopes, or that the responders in this study were exposed to the Msg that is represented in the recombinant fragments or were exposed to a dominant, localized population of P. jiroveci expressing a single Msg. However, the fact that serum samples collected from different global locations, and sera collected locally 10 years apart, exhibit essentially the same patterns of reactivity to the Msg recombinants, supports the former model theory. It has been suggested that Pneumocystis has the potential to evade the immune system by switching the isoform of Msg that is expressed, but in the absence of a continuous culture system for the organism, this theory is difficult to test.

While not wishing to be bound by theory, the inventors' data, as set forth in the Example, suggests that there is a limited number of Msg epitopes that are recognized by serum antibodies, and that exposure to Msg results in cross-reactive antibodies being produced. This is shown by isolating a panel of recombinants that represent different Msgs, and analyzing the patterns of reactivity to those new fragments. Since reactivity to different Msgs can simply mean exposure to those Msgs, and not the presence of cross-reactive antibodies, antibody elution experiments elucidate the extent of cross-reactivity between different isoforms of Msg.

While not wishing to be bound by theory, the inventors believe that the titer of anti-Msg antibodies is important as a measurable parameter of infection. Further, the use of the Msg recombinants would be enhanced by the development of an ELISA system to measure quantitative differences in antibody titer among groups of subjects. Additionally, serum specimens may be obtained over time from patients who do and do not develop episodes of PcP to determine whether epitope recognition and the magnitude of the antibody response can be correlated with susceptibility to and recovery from PcP.

Analysis of the serum samples from four different countries shows that there is no significant difference in recognition patterns of the Msg fragments based on the geographical origin of the sera. These sera had previously shown differential reactivity to various high molecular weight P. jiroveci antigens, including Msg. However, while not wishing to be bound by theory, the inventors believe that the lack of variability in the response to the recombinant Msg fragments suggests that there is a limited number of Msg epitopes that can be seen by serum antibodies.

The ability of the inventors to correlate the reactivity of a given serum sample to a recombinant Msg fragment with its reactivity to the crude preparation of antigen previously analyzed suggests inherent differences in the preparations of antigen. While not wishing to be bound by theory, the inventors believe that the difference in results may reside in the fact that in the current study, the inventors directly analyze the response to recombinant fragments of Msg, whereas previous studies have analyzed the response to a complex mixture of P. jiroveci antigens derived from infected human lungs. Definitive analysis of a singe protein is very difficult when complex mixtures of proteins such as a lung extract are used.

Alternatively, while not wishing to be bound by theory, the inventors believe that the native Msg expressed in P. jiroveci is glycosylated, and the variability in reactivity to the native Msg is due to regional variations in glycosylation of the proteins. The present recombinants are expressed in E. coli and are not post-translationally modified. Therefore, the responses the inventors measure are directed at epitopes in the peptide backbone of Msg and are independent of glycosylation patters. Msg fragments may be engineered for expression in Pichia pastoris, a yeast system that allows glycosylation of expressed proteins. While the glycosylation pattern of P. pastoris may not be the same as that of P. jiroveci, expression of the Msg recombinants in this system enables the analysis of the effect of glycosylation on recognition by serum antibodies. Further, the patterns of serum reactivity to the Msg fragments expressed in two different systems can be compared.

The present data shows that healthy adults have a significantly higher prevalence of serum antibodies to recombinant Msg than HIV⁺ patients, but this frequency of recognition mainly resides in the MsgB fragment. HIV⁺ patients who recovered from PcP recognize MsgC significantly more frequently than HIV⁺ patients who never experienced PcP. Nine HIV⁺ patients who had a serum specimen drawn before they developed PcP did not recognize MsgC. These results are important because they demonstrate how the use of different fragments of a single recombinant antigen can reveal previously unrecognized differences among population groups.

While not wishing to be bound by theory, the inventors believe that it is possible that independent recognition of Msg fragments is of value in analyzing the diversity of epitopes during various stages of HIV infection or following HAART and further, that antibodies play a role in host defenses against PcP in HIV⁺ patients. Although there have been studies in humans, the most direct evidence for the importance of antibodies in the resolution of Pneumocystis infection has come from animal models of PcP. While not wishing to be bound by theory, the inventors believe that their results suggest that the B cell epitopes that elicit these antibody responses may reside in MsgC. Mapping of the epitopes recognized by such antibodies will be facilitated by localization within a single Msg fragment.

### EXAMPLE

Materials and Methods

Cloning, Expression and Purification of Msg Fragments

Oligonucleotides are designed based on the known sequence of P. jiroveci Msg, and are used in PCR to generate three overlapping fragments of a Msg gene. The sequences of the oligonucleotides used are as follows: 5′ AACTACCTTTAAAACTTTCAA 3′ (forward), 5′ TTAGGATCTGGATTCTGA 3′ (reverse) to generate msg₁₅₋₁₁₁₉, 5′ CGAAGCTTATGACTGCGCGAG 3′ (forward), 5′ TCCTCGGAGCTCTACTTTGAG 3′ (reverse) to generate msg₇₂₉₋₂₂₈₂, 5′ TTCCAAAACGCTACGTGTAA 3′ (forward), 5′ TCAATTGATGCTGAAGAGATG 3′ (reverse) to generate msg₂₀₁₅₋₃₃₃₂. The template used to generate msg₇₂₉₋₂₂₈₂ and msg₂₀₁₅₋₃₃₃₂ is a λgt11 clone of human-derived Msg, whereas the template used to make msg₁₅₋₁₁₁₉ is genomic DNA from P. jiroveci-infected human lung.

The sequence of the PCR products is confirmed, and they are cloned into the pET30 expression vector (Novagen, Madison, Wis.) in the correct reading frame and expressed in E. coli. The 3 recombinant fragments are called MsgA, MsgB and MsgC which are encoded by the gene fragments msg₁₅₋₁₁₁₉, msg₇₂₉₋₂₂₈₂ and msg₂₀₁₅₋₃₃₃₂, respectively. Recombinant protein expressed from the pET30 vector without insert is used as a control antigen. The recombinant proteins are expressed in inclusion bodies within E. coli, and are purified using standard methods.

Briefly, bacterial cultures expressing recombinant Msg fragments are harvested by centrifugation, the cell pellet are sonicated and washed three times in binding buffer without urea (5 mM imidazole, 0.5M NaCl, 20 mM Tris-HCl, pH 7.9), and the final pellet is dissolved in binding buffer with 6M urea. The recombinant preparations are purified by affinity chromatography using HIS-binding resin (Novagen, Madison, Wis.) with the urea being removed in the wash stages. Eluted proteins are dialyzed overnight against PBS pH 7.4, filter sterilized and frozen at 70° C. Protein concentration is determined by A₂₈₀ using a standard curve generated with bovine serum albumin.

Western Blot Analysis

Recombinant Msg fragments are run on SDS-PAGE gels (Invitrogen, Carlsbad, Calif.), transferred to nitrocellulose and blocked in 1% non-fat milk in TTBS (20 mM THAM, 0.5M NaCl, 0.05% Tween-20) for 1 hour at room temperature. Blots are cut into strips and individual strips are incubated either overnight at 4° C., or for 2 hours at room temperature, with sera from healthy donors or HIV⁺ patients (1/50 dilution in TTBS), or with a rabbit polyclonal antiserum (1/5000 dilution in TTBS) generated against a cell wall prep of P. jiroveci. The strips are washed three times with TTBS, and incubated either with horseradish peroxidase-labeled goat anti-human IgG or horseradish peroxidase-labeled goat anti-rabbit IgG (Kirkegaard & Perry Laboratories, Gaithersburg, Md.) (1/5000 dilution in TTBS) for 1 hour at room temperature. The strips are washed three times in TTBS, color is developed with TMB membrane substrate (BioFX, Owings Mills, Md.), and the reactions are stopped with H₂O.

To identify the appropriate bands for analysis and to serve as a positive control in our assay, one strip from each gel is blocked in milk as above, probed with horseradish peroxidase-labeled S protein (Novagen, Madison, Wis.) (1/5000 dilution in TTBS) for 1 hour at room temperature, washed three times and color developed as above. S protein reacts with the S•tag which is encoded in the pET30 vector and expressed as a fusion protein in our constructs, and therefore serves as a positive control. It does not react with E. coli proteins. As a negative control, one strip from each gel is blocked in milk as above, incubated with horseradish peroxidase-labeled goat anti-human IgG, washed and developed as above. The strips probed with human serum are compared to the positive and negative control strips. Each assay is read by two independent readers. Results are usually unequivocal, but in cases of ambiguous results, the assay is repeated at least once, and the predominant result from all reads of those samples is reported.

Samples from Volunteers

Samples are obtained from several different sources. Sera from 95 healthy adult blood donors are obtained from the Hoxworth Blood Bank in Cincinnati, Ohio. These samples are stored at −20° C. Four cohorts of serum samples, which had been obtained in 1990 from persons residing in different geographic regions, are also tested. These included sera from HIV seronegative Haitian women involved in a study of transmission of HIV infection; serum samples from South Africa obtained from healthy HIV seronegative men from a rural area; sera from South Korea obtained from HIV seronegative patients attending an outpatient clinic at the University Hospital, College of Medicine, Seoul National University; and serum samples from healthy blood donors from Hoxworth Blood Bank, Cincinnati, Ohio. These geographical samples have previously been tested for reactivity to a crude preparation of P. jiroveci isolated from human lung. Sera from 94 HIV⁺ patients are obtained from banked sera from the University of Cincinnati Infectious Diseases Center. The medical records of these subjects are abstracted to obtain the CD4⁺ lymphocyte counts, HIV RNA levels closest to the time the samples (+/−3 months) and clinical history.

Antibody Elution Assay

Recombinant MsgB is run on SDS-PAGE gels, transferred to nitrocellulose and blocked in 1% non-fat milk in TTBS for 1 hour at room temperature. The location of the recombinant protein is identified by reactivity with S protein, and strips corresponding to the molecular mass of the recombinant are cut out and incubated either overnight at 4° C., or for 2 hours at room temperature, with a rabbit polyclonal antiserum (diluted 1/50 with TTBS) generated against a cell wall preparation of human P. jiroveci. The antiserum is removed and stored, and the strips are washed three times with TTBS. After the final wash, the strips are incubated with 0.1M glycine pH 2.5 for 5 minutes to release the bound antibodies. The released antibodies are removed, and the pH brought to neutrality with Tris-Cl pH 8.5. The blots are rinsed once with TTBS, the antiserum is reapplied and the bound antibodies are eluted as above a total of 3 times. The eluted antibodies are tested for reactivity to a crude preparation of P. jiroveci isolated from infected human lung using Western blot analysis.

Statistics

Statistical analysis is performed using Graphpad Instat (San Diego, Calif.). Sera from healthy blood donors are compared to those with HIV infection to determine the rates of reactivity. Sera from HIV seronegative subjects from Haiti, South Africa, Korea and Cincinnati are compared to determine the rates of reactivity. The inventors calculate that a sample size of 22 subjects per group is expected to have an 80% power to detect a 50% difference in the rate of reactivity (P<0.05). The samples are correlated using a Kappa test. Statistical significance for categorical variables is determined by using a chi-square or Fisher's exact test where appropriate (P<0.05).

Results

Specificity of the Msg Constructs

Using oligonucleotides specific for the sequence of a cloned msg gene, the inventors use PCR to generate 3 overlapping fragments spanning the entire length of the msg gene, illustrated in FIG. 1. Whereas msg₇₂₉₋₂₂₈₂ and msg₂₀₁₅₋₃₃₃₂ are generated using the cloned msg gene as template, msg₁₅₋₁₁₁₉ is generated using P. jiroveci DNA isolated from infected human lung as template. The nucleotide sequence of msg₁₅₋₁₁₁₉ (Genbank AY072779) is 86% identical to the corresponding sequence of the cloned msg gene used as a template for the remaining fragments, and the deduced amino acid sequence is 74% identical to that of the cloned gene. The sequence of msg₁₅₋₁₁₁₉ also exhibits 67-70% identity at the nucleotide level and 57-60% identity at the deduced amino acid level with the corresponding portion of other cloned msg genes, as illustrated in FIG. 2.

The 3 recombinant fragments of Msg (MsgA, MsgB and MsgC) are expressed in E. coli, and are purified away from contaminating bacterial proteins using affinity chromatography. A typical purification profile for MsgB is shown in FIG. 3, with similar profiles being obtained for the other fragments and for the control protein (pET expression vector without insert. Whereas the purification of MsgA and MsgB consistently gives a single band on SDS-PAGE analysis, the profiles for purification of MsgC always has multiple bands in addition to the full length fragment. Presumably these are breakdown products that are co-purified along with the full length fragment because they express the S•tag used for purification. These extra bands are not taken into consideration when scoring reactivity to the MsgC fragment; only reactivity to the full length band is recorded.

To investigate the utility of the recombinant fragments in analysis of human serum antibody reactivity, the inventors demonstrate the specificity of the fragments and that human serum can recognize all 3 fragments. The specificity of the Msg fragments is illustrated in three ways in FIG. 4. First, a polyclonal rabbit antiserum that is raised against a cell wall preparation of human P. jiroveci that includes native Msg, is tested for reactivity to the recombinant fragments in Western blot. All three fragments are recognized by the rabbit antiserum, even though reactivity with MsgC is weak. Second, antibodies from the rabbit antiserum that recognized MsgB are eluted from the Msg fragment and are tested for reactivity to the cell wall preparation used to generate the antiserum. The eluted antibodies show specific reactivity with a protein band whose molecular mass corresponded with that of native Msg and not with any other bands. Finally, both rabbit and human sera (more than 300 human sera tested) show no reactivity to the control protein encoded by the expression vector without insert, showing that the reactivity seen in the inventive assays is directed at the Msg fragments and not at the portion of the recombinants encoded by the expression vector. The reactivity profile of a sample of human serum that recognizes all 3 recombinant Msg fragments but not the control protein, is shown in FIG. 4C.

Recognition of Msg Fragments by Human Serum

The inventors test 95 sera from healthy volunteers for the ability to recognize the Msg constructs by Western blot analysis. Eighty of the 95 samples (84%) recognize at least one of the constructs, whereas 15 (16%) of the sera do not recognize any of the fragments, as depicted in Table 1. Thirty-eight of the serum samples (40%) recognize MsgA whereas 61 sera (64%) and 39 sera (41%) recognize MsgB and MsgC, respectively. Each of the fragments is recognized independently of the others, yielding 8 patterns of recognition. None of the sera tested reacts with the control protein expressed by the empty vector.

Comparison of Sera from Different Geographic Locations

To determine if recognition of the Msg fragments is a function of the geographical source of the serum samples, the inventors test four panels of sera that originated in different geographic locations for the ability to recognize the recombinant fragments. Twenty-two sera each from the US, Haiti, South Africa and South Korea, respectively, are tested by Western blot analysis for the ability to recognize the Msg fragments as depicted in Table 1.

TABLE 1
Recognition of Msg fragments by human immunodeficiency virus
(HIV) negative sera from 4 distinct geographic regions

| | | | | South | South |
|---|---|---|---|---|---|
| | USA^{a} | USA^{b} | Haiti^{b} | Africa^{b} | Korea^{b} |
| Fragment recognized: | (n = 95) | (n = 22) | (n = 22) | (n = 22) | (n = 22) |
| MsgA alone | 7% | 4.5% | 9% | 0% | 0% |
| MsgB alone | 21% | 4.5% | 9% | 14% | 9% |
| MsgC alone | 10% | 9% | 9% | 18% | 4.5% |
| MsgA + MsgB | 15% | 14% | 4.5% | 9% | 0% |
| MsgA + MsgC | 2% | 0% | 9% | 9% | 9% |
| MsgB + MsgC | 13% | 27% | 14% | 14% | 23% |
| MsgA + MsgB + | 16% | 18% | 4.5% | 4.5% | 9% |
| MsgC | | | | | |
| None | 16% | 23% | 41% | 31.5% | 45.5% |
| Total MsgA | 40% | 36% | 27% | 23% | 18% |
| Total MsgB | 64% | 64% | 32% | 41% | 41% |
| Total MsgC | 41% | 55% | 36% | 45.5% | 45.5% |

Data are presented as the % of serum samples in each group that reacted with the indicated Msg fragments.
^{a}Serum samples were collected in 2000, and had not previously been analyzed.
^{b}Serum samples were collected in 1990, and had previously been analyzed against crude preparations of P. jiroveci.

Each of the three Msg fragments can be recognized by sera from each country, and the frequency of recognition did not vary significantly between the groups of sera. Seventeen of 22 sera from the US (77%) react with one or more of the fragments, whereas 59% of Haitian sera, 68% of South African sera and 55% of South Korean sera reacts with one or more of the fragments. Furthermore, the recognition of any one of the eight possible patterns is not statistically different between the panels of serum samples when compared using a Kappa test; for example 36% of US sera (n=8/22), 27% of Haitian sera (n=6/22), 23% of South African sera (n=5/22) and 18% of South Korean sera (n=4/22) recognized MsgA. To determine if the apparent variation in reactivity to the Msg fragments is due to the relatively small number of samples tested, the inventors analyze additional 45 sera from Korea for reactivity to MsgA. When all 67 Korean sera are analyzed together, 39% (26/67) are positive for reactivity to MsgA, compared with 36% and 38% of US sera (1990 and 2000 respectively).

Analysis of HIV⁺ Sera

To determine if the pattern of recognition of the Msg recombinants seen in healthy volunteers (Table 1) is similar in HIV⁺ patients, the inventors assay serum from 94 HIV⁺ donors, illustrated in FIG. 5A The median CD4⁺ lymphocyte count within 3 months of the collection of sera is 94 cells/mm³ (n=86). The median HIV-1 RNA level is 87,000 copies/ml (n=67). Few subjects, 4.4% (n=3/67), with HIV infection have an HIV-1 RNA level <400 copies/ml. Thirty-five subjects have a prior episode of histologically proven PcP.

The overall reactivity of the HIV⁺ sera is lower than the healthy sera, in that 32/94 of the HIV⁺ sera (34%) do not react with any of the fragments, whereas only 15/95 (16%) of the healthy sera showed lack of reactivity (P=0.003). The reactivity to individual fragments of Msg varies depending on the source of the serum, and the identity of the fragment being tested. Both MsgA and MsgB are recognized more frequently by healthy sera than by HIV⁺ sera. MsgA is recognized by 38 out of 95 healthy sera (40%) but by only 25/94 HIV⁺ sera (P=0.064). However, the difference in recognition of MsgB is significant in that 61/95 of healthy sera (64%) and 42/94 of HIV⁺ sera (45%) recognize the fragment (P=0.0086). There is no difference in frequency of recognition of MsgC as 39/95 healthy and 37/94 HIV+sera (41% and 39% respectively) recognize the fragment.

The reactivity of HIV⁺ sera to individual fragments of Msg varies depending on whether the patient had a previous bout of PcP (FIG. 5B. Twenty of 34 PcP+patients (59%) recognize MsgC compared with only 28% of 60 PcP⁻ patients (P=0.0046). Of particular interest, none of the 0.9 HIV⁺ patients who went on to develop PcP after their serum specimen is obtained had antibodies to MsgC. There is no significant difference in the rates of reactivity to the other Msg fragments between the groups of sera (FIG. 5B. There is also no significant difference in frequency of reactivity to any of the fragments when the HIV⁺ sera are separated based on CD4⁺ T cell count (comparing those with <100 CD4⁺ T cells/mm³ to those with >100 CD4⁺ T cells/mm³) or when separated based on viral titer (comparing those with <100000 viral copies/ml to those with >100000 viral copies/ml). The data provided in the Example illustrates that these novel recombinant Msg fragments are useful for analyzing antibody response in an individual.

What we claim is:

1. A method of analyzing antibody response in an individual to major surface glycoprotein (Msg), comprising analyzing a pattern of reactivity of a sample with a recombinant Msg fragment.

2. The method according to claim 1, wherein the recombinant Msg fragment comprises Msg A, Msg B, Msg C, or a combination thereof.

3. The method according to claim 1, wherein the sample comprises blood sample, tissue sample, serum samples or combinations thereof.

4. The method according to claim 1, wherein the individual is healthy or HIV⁺.

5. The method according to claim 3, wherein the HIV⁺ individual has had a Pneumocystis pneumonia infection.

6. The method according to claim 1, wherein the step of analyzing the pattern of reactivity comprises:
a. determining the pattern of reactivity exhibited by the sample with the Msg fragment; and
b. comparing the pattern of reactivity exhibited to a predetermined pattern of reactivity.

7. The method according to claim 1, wherein the sample is from an individual having a Pneumocystis pneumonia infection.

8. The method according to claim 1, wherein the sample is from an individual in convalescence following an episode of Pneumocystis pneumonia infection.

9. The method according to claim 1, wherein the sample is from an HIV⁺ individual following highly active anti-retroviral therapy (HAART).

10. An immunological assay for analyzing antibody response to major surface glycoprotein (Msg) in an individual comprising recombinant Msg fragments.

11. The immunological assay according to claim 10, wherein the recombinant Msg fragments comprise MsgA, MsgB, MsgC or combinations thereof.

12. A recombinant MsgA fragment.

13. A recombinant MsgB fragment.

14. A recombinant MsgC fragment.


SEQUENCE LISTING (13 sequences)

SEQ ID NO: 1 - protein, 336 aa, Human
LARAVARAVKRRGAAAQGTSVYNDEEILLALILKEDGLEEKKCKEKLKRYCEALKNVTLTADKIHEKLKDFCNNGNEEKKCKGLKDKIEKKCNDFKKDKLEKKLTNPSDNDCKEHERQCLFLEGACPSDLIENCNKLRNKCYQKKRDRVAEEVLLRALRGDLEKETECEKKIKDVCPKIGQESDELTLLCLDQKKTCMNLITAREKKCNKLEEDVKKALENKNNLLGKCLPLLEQCYFHRGDCKKKASQCKPPNKDCEDYLPKCDELAEECGKKGIIYIHPGPDFDPTKPEPTVAEDIGLEELYKKAAEDGVHIGKPPVRDATALLALLIQNPDPK

SEQ ID NO: 2 - protein, 329 aa, Human
VARAVKRQVAGVKNNEAEERLFALIMRADYKDESKCKNKIKEYCDGLKNASLTSEEVHKELKDFCKDGSQGKKCEELKKNVEAKCNNFKTKLEGLVKKDASGLTNDDCKENERQCLFLEGACPDLVEDCSKLRNLCYQKKREGVAEEVLLRALRGDLGNKTECEKKIKDVCPKIGQESDELTLLCLDQKKTCTNLMTARDKKCNTLEEDVKKALENKNNLLGKCLPLLEHATFTEGTAKKASQCTPNKDCEDYLPKCDELAEECGKKGIIYIHPGPDFDPTKPEPTVAEDIGLEELYKKAAEDGVHIGKPPVRDATALLALLIQNPDPK

SEQ ID NO: 3 - protein, 331 aa, Human
ARAVKRRAKGAQNSIDEEHVLALILKKNGLEDTKCKTKLEEYCKTLTNAGLNPEKVHEKLKDFCDNGKRNEKCQDLKNKVNQKCIKFQGKLQTAAGKKISELTDEDCKKNEQQCLFLEGACPTELKDDCNKLRNNCYQKERNNVAEEVLLRALRGDLNETKTCEKKLKEVCPKLERESDELTELCLYQKTTCVSLVTKGKSKCDTLEKEVEEALKKNELREKCLLLLEQCYFHRGNCEGDKSKCNKPNNKDCKEYVPECDELAEKCGKENIVYMHPGSDFDPTKPEPTLAEDIGLEELYKRAEEDGIFVGRQHVRDATALLALLLKKTLKK

SEQ ID NO: 4 - protein, 331 aa, Human
ARAVKRQAKGAQNSIDEEHVLALILKKNGLEDTKCKTKLEEYCKTLTNAGLNPEKVHEKLKDFCDNGKRNEKCQDLKNKVNQKCIKFQGKLQTAARKKISELTDEDCKKNEQQCLFLEGACPTELKDDCNKLRNNCYQKERNNVAEEVLLRALRGDLNETKTCEKKLKEVCPKLERESDELTELCLYQKTTCVSLVTKGKSKCDTLEKEVEEALKKNELREKCLLLLEQCYFHRGNCEGDKSKCNKPNNKDCKEYVPECDELAEKCGKENIVYMHPGSDFDPTKPEPTLAEDIGLEELYKRAEEDGIFVGRQHVRDATALLALLLKKTLKK

SEQ ID NO: 5 - protein, 279 aa, Human
KPEKVHKKLKEFCENKKADSKCKELKEKLTQKCTAIKGKLTEAIKKKNSDLTDEDCKENEQQCLFLEGACPAELKDDCNTLRNKCYQKKRDKVAEEALLRAVRGGLINETTCEGKLKEVCIELSQESDELTKLCLYQKMTCKTFVLEKQKKCNALKQDVNAALEKKDELRGKCLPLLERCYFYRGNCEDISKCNKSSEDCYEYLPVCDTLAVKCEENKIIYTHPGSDFNPTKSKPTVAEDIGLEELYKKAAEEGVHIGKPPVRDATALLALLIQNLDPK

SEQ ID NO: 6 - protein, 316 aa, Human
ARAVKRQVTGASGQYDDEVNILALILQEDAMEDTKCKKSLEKYCEELKKASLDMEKVHKMLKDFCGNGKASKANTKCQGLQAKVTGKCTNFKTQKLGPALTNPSDDNCKESERQCLFLEGACHNLVEDCNKLRNLCYQKKRDGVAEEVLLRALRSDLNKTETHEKKLKEICPVLQRESNELTDLCLNQKKTCENIIKEKDKKCTTLKANVATALGSFKKEICLELLEQCYFYIGNCGDDDIIKCIELGGKCQEQNIVYIPPGPDFDPTRPEATLAEDIDLDELYKKAEEDGVFIGKHHLRDATALLTLLVKKDDTG

SEQ ID NO: 7 - protein, 316 aa, Human
ARAVKRQAAGTQNSIDEEHVLALILKEDGLSEQECKKKLKKYCQELTEAKLNIEQVHRKLKGFCEDGKADTKCKELKANIEKKCTTIKGKLKEAIKKKIQIITDKDCKENEQQCLFLEGVCSKELKDDCNTLRNKCYQKKRDKVAEEVLLRALRSDLNGSVICEKKLKEICPVMGRESDELTNLCLNQKETCKNILIEKDKKCGTLKTDVSAALGSFKKETCLELLEQCYFYIGNCGDDDIIKCIELGGKCQEQNIAYMPPGPDFDPTRPEATIAEDIGLEEFYKKVEEDGVFIGKNHLRDATALLALLIQDSSLK

SEQ ID NO: 8 - DNA, 21 bp, Human
aactacctttaaaactttcaa

SEQ ID NO: 9 - DNA, 18 bp, Human
ttaggatctggattctga

SEQ ID NO: 10 - DNA, 21 bp, Human
cgaagcttatgactgcgcgag

SEQ ID NO: 11 - DNA, 21 bp, Human
tcctcggagctctactttgag

SEQ ID NO: 12 - DNA, 20 bp, Human
ttccaaaacgctacgtgtaa

SEQ ID NO: 13 - DNA, 21 bp, Human
tcaattgatgctgaagagatg